(19) United States
(12) Patent Application Publication
(10) Pub. No.: US 2022/0016423 A1
(43) Pub. Date: Jan. 20, 2022

(54) BRAIN INTERFACING APPARATUS AND METHOD

(71) Applicant: BRAINPATCH LTD, London (GB)

(72) Inventors: Nickolai Vysokov, London (GB); Illya Tarasenko, London (GB); Dauren Toleukhanov, London (GB); Mikheil Oganesyan, London (GB)

(21) Appl. No.: 17/413,310

(22) PCT Filed: Dec. 13, 2019
(86) PCT No.: PCT/IB2019/060767

§ 371 Date: Jun. 11, 2021

(30) Foreign Application Priority Data
Dec. 14, 2018 (GB) 1820468.5

(51) Int. Cl. A61N 1/36; A61N 1/04; A61N 1/02

(52) U.S. Cl. CPC A61N 1/36031; A61N 1/0456; A61N 1/0484; G06F 3/015; A61N 1/36025; A61N 1/025; A61N 1/0476

(57) ABSTRACT

Here is disclosed brain interfacing apparatus that provides, when in operation, brain activity monitoring and stimulation of brain of user comprising headwear arrangement to be placed or positioned on head of user wherein headwear arrangement comprises electrode arrangement including plurality of electrodes that makes electrical contact with scalp of user, input/output arrangement that receives electrical signals from plurality of electrodes and delivers brain stimuli using brain stimulation protocol to plurality of electrodes, data processing arrangement that processes detected electrical signals received from input/output arrangement and generates brain stimulation protocol corresponding to received electrical signals, wherein data processing arrangement includes memory module; and power units that supply electrical power to input/output arrangement and data processing arrangement. Data processing arrangement compares received electrical signals with predetermined reference data set to generate analysis of received electrical signals and applies, machine learning algorithm or another computational algorithm to analysis when generating brain stimulation protocol.

### TECHNICAL FIELD

The present disclosure relates to neuromodulation devices, and to methods of using aforesaid neuromodulation devices. More particularly, the present disclosure relates to brain interfacing apparatus and methods for using such apparatus, for example by employing artificial intelligence (adaptive learning) implemented using computing arrangements that modify a manner of operation of the brain interfacing apparatus when processing signals therethrough, when in operation. Additionally, the present disclosure is concerned with computer programme products comprising a non-transitory computer-readable storage medium having computer-readable instructions stored thereon, the computer-readable instructions being executable by a computerised device comprising processing hardware to execute the aforesaid methods.

### BACKGROUND

Recently, equipment that is operable to stimulate human nervous systems has significantly evolved. However, electrochemical signals originating in a given brain of a given person are isolated from external influences by a skull of the given brain, wherein the electrochemical signals are spread spatially by the skull. Moreover, Non-Invasive Brain Stimulation (NIBS) systems are being contemporarily used for stimulation of brains. In an example, a function of a nervous system is modified by applying electrical stimulation to the nervous system to control a perception of pain by the nervous system, or a different brain stimulation protocol is used to enhance performance of the nervous system when performing cognitive tasks.

Typically, electrochemical signals originating in the given brain of the given person are detected by connecting electrodes in contact with a scalp region of the given person. Generally, signals picked up from the given brain, by using electrodes connected in contact with the scalp region, have an amplitude in an order of tens to hundreds of microvolts. These signals or parameters derived from these signals are linked to various brain states, cognitive activity and particular disorders. It is also possible to use these same or different electrodes to deliver electrical currents for Non-Invasive Brain Stimulation (NIBS). Moreover, a majority of conventional Non-Invasive Brain Stimulation (NIBS) systems are relying on a “one-fits-all” protocol, namely a common protocol used generally for different types of brains. Conversely, human brains are found to be highly individualistic, namely mutually different from one another, in a manner in which they respond to stimuli. Moreover, a rigid conventional system of electrode positioning (also known as the 10/20 system) that takes into account the size of the skull and is used for both electrical recording and electrical stimulation relies on assistance from a nurse or a technician, but it still doesn't take into account the individualities of the brain and algorithms of signal processing and stimulation that do not adapt can thus lead to poor reproducibility and unexpected outcomes in extreme cases. Therefore, such a single design of apparatus pursuant to the “one-fits-all” protocol is a crude and ineffective approach.

Such individualistic requirements highlight a major challenge and need in the field for optimising stimulation with respect to inter-individual structural variabilities, but also with respect to individual signalling dynamics and even with respect to the quickly changing state of the brain in real time. As the brain stimulation affects a state of the brain, the stimulation potentially needs to be adapted accordingly, thus creating a “feedback loop”. If that adaptation happens automatically in real-time without involvement of any third party, this type of loop is known as a “closed loop”. If the stimulation were not effective at changing the state of the brain towards a desired state, then the collective parameters of the stimulation, which can be defined as the “brain stimulation protocol”, would need to be adjusted in this feedback loop until the desired effect is achieved. Thus, we define any arrangements for processing the incoming signal or for adjusting the brain stimulation protocol, which can adapt to the inter-individual and inter-state differences, as “adaptive learning algorithms”.

However, majority of existing specialised devices are limited in their ability to provide a truly closed-loop Non-Invasive Brain Stimulation (NIBS). Notably, the existing specialised equipment lacks real-time protocols for adjusting and optimising stimulation, resulting in poor reproducibility of the beneficial effects that Non-Invasive Brain Stimulation (NIBS) is capable of providing. Moreover, most of the efforts made in this direction have so far been focused predominantly on triggering the stimulation in response to a positive or negative phase of the recorded brain waves. For example, to trigger a stimulation in phase with recorded brainwaves of a given subject person, a form of “phase-locking” is contemporarily used, as described in a WIPO publication WO2017015428A1.

Despite advancements that have been made in the aforementioned neuromodulation equipment, stimulation parameters need to be further optimised to achieve an improved real-time optimisation of Non-Invasive Brain Stimulation (NIBS). Therefore, in light of the foregoing discussion, there exists a need to overcome the aforementioned drawbacks associated with conventional Non-Invasive Brain Stimulation (NIBS) systems.

### SUMMARY

The present disclosure seeks to provide an improved brain interface apparatus, for example a NIBS apparatus, that is better able to adapt its stimulation parameters to individual requirements and characteristics of each person to which the apparatus is applied.

Moreover, the present disclosure seeks to describe an improved method for using the improved brain interface apparatus, for example a NIBS apparatus, that is better able to dynamically adapt its stimulation parameters to the requirements of an individual and characteristics of each person to which the apparatus is applied, depending on the response of the individual's brain to the stimulation.

An objective of the present disclosure is to provide a solution that overcomes at least partially the problems encountered in NIBS in prior art, and provides an improved brain interface apparatus to users.

In a first aspect, embodiments of the present disclosure provide a brain interfacing apparatus that runs, when in operation, brain activity monitoring and stimulation of a brain of a user, wherein the apparatus comprises:

(i) a headwear arrangement to be placed or positioned on a head of the user wherein the headwear arrangement comprises an electrode arrangement including a plurality of electrodes that makes electrical contact with a scalp of the user, when in operation, to detect electrical signals therefrom and to apply brain stimuli thereto;
(ii) an input/output arrangement that receives electrical signals from at least one of the plurality of electrodes and delivers the brain stimuli using at least one of the plurality of electrodes, when in operation;
(iii) a data processing arrangement that processes the detected electrical signals received from the input/output arrangement and generates a brain stimulation protocol, which depends on the received electrical signals, when in operation, wherein the data processing arrangement includes a memory module; and
(iv) a power unit that supplies electrical power to the input/output arrangement and the data processing arrangement,
characterised in that the data processing arrangement compares the received electrical signals with a predetermined reference data set to generate an analysis of the received electrical signals and applies at least one adaptive learning algorithm or another computational algorithm to the process of analysing and generating the brain stimulation protocol. The present disclosure is of advantage in that it provides a personalised brain interfacing apparatus capable of providing user-specific stimulation in an adaptive and real-time manner, thus, resulting in a user-friendly stimulation environment for achieving desired effects.

Embodiments of the disclosure are advantageous in terms of providing a brain interfacing apparatus, which has the potential for amelioration of symptoms associated with insomnia, attention deficit hyperactivity disorder, epilepsy and tremor in Parkinson's disease through neuromodulation optimised to individual brain signalling dynamics. Furthermore, the apparatus of the present disclosure provides a solution for achieving safe and effective transcranial stimulation, non-invasive recording of brain activity and real-time optimisation of brain stimuli in accordance with the response received from the brain.

In a second aspect, embodiments of the present disclosure provide a method for using a brain interfacing apparatus that provides, when in operation, brain activity monitoring and stimulation of the brain of a user, including:

(i) using a power unit to supply electrical power to an input/output arrangement and a data processing arrangement;
(ii) placing or positioning a headwear arrangement on the head of the user, wherein the headwear arrangement comprises an electrode arrangement including a plurality of electrodes that makes electrical contact with the scalp of the user, when in operation, to detect electrical signals therefrom and to apply brain stimuli thereto;
(iii) using the input/output arrangement to receive electrical signals from at least one of the plurality of electrodes and to deliver the brain stimuli to at least one of the plurality of electrodes;
(iv) using the data processing arrangement to process the detected electrical signals received from the input/output arrangement and to generate the brain stimulation protocol optimised with respect to the received electrical signals, wherein the data processing arrangement includes a memory module; and
(v) comparing the received electrical signals and a predetermined reference data set for generating an analysis and applying at least one adaptive learning algorithm or another computational algorithm to the analysis for generating the brain stimulation protocol.

In a third aspect, embodiments of the present disclosure provide a computer programme product comprising a non-transitory computer-readable storage medium having computer-readable instructions stored thereon, the computer-readable instructions being executable by a computerised device comprising processing hardware to execute the aforementioned method.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary embodiments of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 is a schematic illustration of a block diagram of a brain interfacing apparatus for brain activity monitoring and stimulation of the brain of a user, in accordance with an embodiment of the present disclosure;

FIGS. 2A and 2B are illustrations of exemplary implementations of the brain interfacing apparatus of FIG. 1 applied on a user, in accordance with an embodiment of the present disclosure;

FIG. 3 is an illustration of a closed loop system for implementing at least one adaptive learning algorithm, in accordance with an embodiment of the present disclosure;

FIG. 4 is an illustration of an exemplary implementation of the brain interfacing apparatus comprising a control unit and an external stimulation arrangement, in accordance with an embodiment of the present disclosure;

FIG. 5 is an illustration of an exemplary implementation of the brain interfacing apparatus comprising an external stimulation arrangement, in accordance with an embodiment of the present disclosure;

FIG. 6 is an illustration of an exemplary implementation of the brain interfacing apparatus with a different headwear arrangement, in accordance with an embodiment of the present disclosure;

FIG. 7 is an exemplary user interface for receiving instruction from a user or for displaying the personalized brain stimulation protocol applied to the user, in accordance with an embodiment of the present disclosure;

FIGS. 8A-B show spectrograms and of signals detected from O1 (channel 7 and channel 8 respectively) region of a brain of a user, in response to various stimulation frequencies used for determination of an optimal stimulation frequency for a user, in accordance with an embodiment of the present disclosure;

FIG. 9 shows a graph illustrating a non-linear relationship between stimulation frequency delivered by LEDs and response power of brain signal with frequency corresponding to stimulation frequency with LED light, in accordance with an embodiment of the present disclosure; and

FIG. 10 is an illustration of steps of a method for (of) brain activity monitoring and stimulation of the brain of a user, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item to which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognise that other embodiments for carrying out or practising the present disclosure are also possible.

In one aspect, an embodiment of the present disclosure provides a brain interfacing apparatus that provides, when in operation, brain activity monitoring and stimulation of the brain of a user, wherein the apparatus comprises:

(i) a headwear arrangement to be placed or positioned on the head of the user wherein the headwear arrangement comprises an electrode arrangement including a plurality of electrodes that makes electrical contact with the scalp of the user, when in operation, to detect electrical signals therefrom and to apply brain stimuli thereto;
(ii) an input/output arrangement that receives electrical signals from at least one of the plurality of electrodes and delivers the brain stimuli to at least one of the plurality of electrodes, when in operation;
(iii) a data processing arrangement that processes the detected electrical signals received from the input/output arrangement and generates the brain stimulation protocols, which are dependent on the received electrical signals, when in operation, wherein the data processing arrangement includes a memory module; and
(iv) a power unit that supplies electrical power to the input/output arrangement and the data processing arrangement, characterised in that the data processing arrangement compares the received electrical signals with a predetermined reference data set to generate an analysis of the received electrical signals and applies at least one adaptive learning algorithm or another computational algorithm to the process of analysing and generating the brain stimulation protocol.

In another aspect, an embodiment of the present disclosure provides a method for using a brain interfacing apparatus that provides, when in operation, brain activity monitoring and stimulation of the brain of a user, characterised in that the method includes:

(i) using a power unit to supply electrical power to an input/output arrangement and a data processing arrangement;
(ii) placing or positioning a headwear arrangement on the head of the user, wherein the headwear arrangement comprises an electrode arrangement including a plurality of electrodes that makes electrical contact with the scalp of the user, when in operation, to detect electrical signals therefrom and to apply brain stimuli thereto;
(iii) using the input/output arrangement to receive electrical signals from at least one of the plurality of electrodes and to deliver the brain stimuli to the at least one of the plurality of electrodes;
(iv) using the data processing arrangement to process the detected electrical signals received from the input/output arrangement and to generate the brain stimulation protocol dependent on the received electrical signals, wherein the data processing arrangement includes a memory module; and
(v) comparing the received electrical signals and a predetermined reference data set for generating an analysis and applying at least one adaptive learning algorithm or another computational algorithm to the analysis for generating the brain stimulation protocol.

The present disclosure provides the aforementioned apparatus and the aforementioned method for providing brain activity monitoring and stimulation, when in operation. The device described herein is simple, robust, inexpensive, and allows for providing electrical stimuli in an efficient manner. The apparatus efficiently senses the brain activity, and provides brain stimuli as a feedback therefrom, in a manner that is robust, effective, and adaptive.

Throughout the present disclosure, the term “user” as used herein relates to any person (i.e., human being) using the aforesaid apparatus. Optionally, the user may be a person having a certain physical or mental disorder such as epilepsy, a head injury, encephalitis, brain tumour, encephalopathy, memory related problems, sleep disorders, stroke, dementia etc.

Alternatively, the user may be a person willing to achieve a specific state of mind, such as an enhanced concentration, relaxation, mental capabilities or, in general terms, enhanced performance for executing a task.

Throughout the present disclosure, the term “brain activity monitoring” as used herein relates to monitoring of electrical signals received from the brain by a method of electroencephalography (EEG). Optionally, the brain activity monitoring may include detection of signals which include, but are not limited to, signals, or a combination of signals, obtained using electric field encephalography (EFEG), Near infrared spectroscopy (NIRS), Magnetoencephalography (MEG), Electromyography (EMG) including signals coming from electrodes located spatially remote from the given user's scalp, Electrocardiography (ECG), eye tracking and/or Functional magnetic resonance imaging (fMRI). More optionally, the brain activity monitoring relates to monitoring of a change in electrical activity of the brain of a user, upon providing external electrical stimulus to the brain of the user. More optionally, the electrical activity of the brain of a user may be indicative of biological parameters related to the mental and physical health of a user including, but not limited to, a heart rate, a breathing rate and a skin conductance.

Throughout the present disclosure, the term “brain stimulus” or “brain stimuli” (plural of “stimulus”) as used herein relates to an external electrical current or to a defined sequence or multiple sequences of electric current amplitudes between a pair, several pairs or any combination of the electrodes applied to the scalp of a user or to locations spatially remote from the scalp of a user, in order to modify and/or enhance an electrical activity in the brain of the user or in the nervous tissues that the current is able to reach. Moreover, in an example, brain stimuli applied to the scalp of the user are analogue external electrical signals having a voltage in a range of 1 millivolt to 50 volts and having a current in a range of 0.1 milliampere to 20 milliamperes.

Throughout the present disclosure, the term “stimulation” as used herein relates to altering (referring to raising, lowering or otherwise modulating) levels of physiological or nervous activity in the brain or in the tissues spatially remote from the given user's brain. Notably, the stimulation of the brain of the user is carried out with help of electrical signals, applied to the scalp of the user with the help of one or more electrodes. Further, stimulation of the brain is achieved by using any one of minimally invasive brain stimulation or non-invasive brain stimulation methods, or optionally both.

Throughout the present disclosure, the term “electrodes” as used herein relates to one or more electrical conductors, with the materials of these conductors including, but not limited to stainless steel, platinum, sliver chloride-coated silver, carbon rubber, graphene and other metamaterials, as well as hydrogels, silicone, sponges, foam or any absorbent with a conducting medium, where necessary to be placed between the conductors and the scalp or skin, including, but not limited to electrically conductive gels and pastes (such as Ten20 paste), as well as liquids (such as physiological saline solution) with such an ionic composition as to establish an electrical path to detect electrical signals generated by the neurons inside the brain and to provide brain stimuli to the neurons and/or other cells present inside the brain of the user. Furthermore, the electrodes are operable to convert an ionic potential into an electric potential and to induce electromagnetic fields on the scalp and inside the skull. Moreover, the electrodes can be of minimally invasive (such as needle electrodes or micro electrodes) or non-invasive type (such as surface electrodes), or optionally both. In an example, the electrodes comprise an assembly of saline-soaked foam, conductive carbon and a metal contact. In such an example, the metal contact is operatively coupled with one or more components of the brain interfacing apparatus (such as, an input/output arrangement and/or a data processing arrangement, described in detail herein later).

The brain interfacing apparatus pursuant to the present disclosure comprises a headwear arrangement including the plurality of electrodes. In use, the plurality of electrodes is placed or positioned on the scalp of the user, in order to establish an electrical contact with neurons in the brain of the user. Such electrical contact establishes an electrical path to detect electrical signals generated by the neurons and to provide brain stimuli to the neurons and/or other cells present inside the brain of the user. The plurality of electrodes detects the electrical signals generated inside the brain of the user by activity of neurons, wherein the detected electrical signals are provided to the input/output arrangement. Generally, the amplitude of the detected electrical signals ranges between 1 microvolt to 100 microvolts. The plurality of electrodes may optionally be configured as any suitable EEG electrode arrangement known in the art. The plurality of electrodes are hybrid electrodes which can function as both for EEG recording and/or for electrical stimulation, for example, transcranial current stimulation (tCS), transcranial direct current stimulation (tDCS), transcranial alternating current stimulation (tACS), transcranial random noise stimulation (tRNS), transcranial temporal interference stimulation (TI), transcranial temporal summation (TS) and/or any other arbitrary transcranial electric current stimulation protocol generated by the adaptive algorithms (tES). A plurality of magnetic coils can be used instead of electrodes to deliver transcranial static magnetic field stimulation (tSMS), low field magnetic stimulation (LFMS), repetitive transcranial magnetic stimulation (rTMS) and/or any other arbitrary transcranial magnetic stimulation (TMS) protocol generated by the adaptive algorithms. Alternatively, also, a plurality of ultrasound generators can be used for delivery of Focused Ultrasound Stimulation (FUS) protocols also generated by the adaptive algorithms. Throughout the present disclosure, the terms “headwear” or “headwear arrangement” as used herein relate to an element of clothing which is worn by the user on his/her head. Optionally, the headwear arrangement may include, but not be limited to, any one of a cap, a hat, a helmet, headphones, a headband, glasses or a bonnet. More optionally, the headwear arrangement may be fabricated in a manner such that it comprises a layer of electrically insulating material. In an example, the headwear arrangement can be fabricated from one of materials including, but not limited to, wool, cotton, polyester, rubber, lycra, nylon or buckram.

Throughout the present disclosure, the term “input/output arrangement” as used herein relates to programmable and/or non-programmable components that, when in operation, receive, modify, convert, process or generate one or more types of signals. Optionally, the input/output arrangement is implemented as a hardware or a software, or a combination thereof.

Throughout the present disclosure, the term “data processing arrangement” as used herein relates to programmable and/or non-programmable components that, when in operation, execute one or more software applications for storing, processing and/or sharing of data and/or a set of instructions. Optionally, the data processing unit can include, for example, a component included within an electronic communications network. Furthermore, the data processing arrangement may include hardware, software, firmware or a combination of these, suitable for storing and processing various information and services accessed by the one or more user using the one or more user equipment. Optionally, the data processing arrangement may include functional components, for example, a processor, a memory, a network adapter and so forth. For example, the data processing arrangement can be implemented using a computer, a phone (for example, a smartphone), a local server, a server arrangement (such as, an arrangement of two or more servers communicably coupled with each other), a cloud server, a quantum computer and so forth. Throughout the present disclosure, the term “memory module” as used herein relates to a volatile or persistent medium, such as an electrical circuit, magnetic disk, virtual memory or optical disk, in which a computer and/or a data processing arrangement may store data for any duration. Optionally, the memory module may be a non-volatile mass storage such as physical storage media.

Throughout the present disclosure, the term “power unit” as used herein relates to a power source being configured to provide electrical power to one or more components of the brain interfacing apparatus. Optionally, the power unit may include one or more cells or batteries capable of providing electrical power. In an example, the power unit may provide 12 Volts electrical supply to a stimuli generator in the input/output arrangement and 5 Volts electrical supply to the data processing arrangement. Optionally, the power unit may also include a power boost generator and regulator circuitry to turn a 3.7 V supply from a battery into a 5 V supply for the brain interfacing apparatus and a 12-40 V supply for the stimuli generator. Optionally, the power unit may also contain circuitry that includes a voltage splitter to provide +/−12-40 V to the stimuli generator.

Throughout the present disclosure, the term “predetermined reference data set” as used herein relates to data derived from EEG recordings from a plurality of persons. Further, the plurality of persons may be of various age group, sex, mental and physical health condition, and geographical location.

Throughout the present disclosure, the term “adaptive learning algorithm” as used herein relates to software-based algorithms that are executable on computing hardware and are operable to adapt and adjust their operating parameters depending upon information that is presented to while trying to minimize a predefined error/loss metric, or processed by, the software-based algorithms when executed on the computing hardware.

Throughout the present disclosure the term “real-time” refers to any process or a set of processes that are being executed concurrently or in a temporally alternating manner with a small time lag in between these alternations. Moreover, where a set of processes must be executed in a sequential manner, the term “concurrently” would refer to the processes being executed in parallel with a minimal delay/time-shift relative to each other.

Throughout the present disclosure, the term “brain stimulation protocol” as used herein, refers to an electrical signal containing information about brain stimuli to be generated. It should be noted that in an embodiment of the present invention where the plurality of electrodes includes electrodes placed at locations remote from the given user's scalp, the brain stimulation protocol may also include information about the stimuli to be generated at these electrodes. It should also be noted, that the brain stimulation protocol also refers to the information that may change throughout the duration of the stimulation as a result of the process of optimisation described in the present disclosure. For example, the information includes one or more electrical characteristics for each electrode, such as an amplitude, a time-period, a phase, one or more frequencies and the power of these frequencies giving rise to a specific sequence of brain stimuli to be generated. The generated brain stimuli will be in the form of a defined sequence or multiple sequences of electric current amplitudes between a pair, several pairs or any combination of the electrodes. Optionally, the brain stimulation protocol includes the time duration for which brain stimuli have to be applied to the scalp of the user. Optionally, the brain stimulation protocol refers to information about at least one of: a visual stimulation, an audio stimulation and/or a virtual reality stimulation to be generated and provided to the user.

Optionally, the plurality of electrodes may include separate electrodes configured for EEG recording and electrical stimulation respectively. Alternatively, the electrode arrangement may include a separate electrode for each location at which it may be desirable to detect EEG signals and/or provide electrical stimulation.

In an embodiment, the plurality of electrodes is in electrical contact with an appreciable area of a given user's scalp; for example, the electrodes may be user-replaceable electrodes and may be lightly spring-loaded to provide a positive contact onto the user's scalp when the headwear arrangement is worn by the user. More optionally, the end of the electrodes may include a 2-D array of small pointed sub-electrodes modified with conducting medium to safely deliver sufficient current, wherein the end could have an area of any size including, but not limited to 4 mm×4 mm, but other appropriate areas could be used, and the sub-electrodes are pointed and can find a path between hairs of the scalp to make contact onto skin of the scalp. Specifically, the plurality of electrodes is spatially located such that the voltage applied across the electrodes generates the electromagnetic field in specific parts of the brain.

Furthermore, the plurality of electrodes, when actively delivering current and when in contact with the scalp of the user, apply electromagnetic fields to the brain of the user acting as brain stimuli. Such brain stimuli are provided with the help of generated brain stimulation protocols received by the input/output arrangement from the data processing arrangement. The generated brain stimulation protocols received from the data processing arrangement are processed by the input/output arrangement, namely converted, into an analogue form and adjusted to a desired current amplitude, before being applied as brain stimuli to the scalp of the user.

In an embodiment the plurality of electrodes used for providing the brain stimuli to the brain of the user, may be arranged in one pair, in more than one pair or in any combination of stimulating electrodes as determined by the brain stimulation protocol.

The input/output arrangement includes an input signal processing arrangement comprising a pre-processor and an input converter. The input signal processing arrangement, when in operation, processes and/or modifies electrical signals received from the brain of the user. Optionally, the pre-processor includes an amplifier, more specifically it may include a programmable gain amplifier, which stabilises the electrical signals received from the brain and amplifies the signals by an amplification factor in a range of 2× to 100× for obtaining an amplified signal, wherein 2× amplification factor is used for a very high dynamic range of analogue to digital conversion for the option of digital pre-processing and artefact subtraction. Optionally, the pre-processor may include one or more analogue filters (such as an electrical noise filter or the stimulation artefact filter) to reduce specific artefacts and/or noise. The electrical signals received form the brain are time-varying, namely are analogue in nature. However, the data processing arrangement only understands (namely, processes) digital bits, therefore it is essential to convert the received electrical signal (analogue in nature) from the brain to digital bits, so that the data processing arrangement is able to understand (namely, process) the received electrical signals from the brain after analogue to digital conversion. The input converter receives the amplified signal and converts it into a form suitable for analysing and processing. Furthermore, the input converter includes an analogue-to-digital converter. In an example, the input signal processing arrangement receives analogue electrical signals having an amplitude in a range of 1 microvolt to 12 Volts from the scalp of the user and the pre-processor eliminates some artefacts and noise and amplifies the signals to generate corresponding amplified signals having amplitudes in a range of up to 12 V. Subsequently, the amplified signals are converted into corresponding digital signals having a sequence of discrete values representative of the corresponding range.

The input/output arrangement further includes an output converter and a stimuli generator. In operation, a brain stimulation protocol is received from the data processing arrangement which is communicably coupled with the input/output arrangement. The received brain stimulation protocol is in the form of digital or discrete signal. Furthermore, the received brain stimulation protocol is sent to the output converter wherein, the output converter converts the received brain stimulation protocol into an analogue signal having varying voltage amplitude with respect to time. The stimuli generator receives the converted analogue signals from the output converter and may optionally convert the set voltage signals into defined current signals. The output of the stimuli generator is acting as brain stimuli and the generated brain stimuli are applied to the scalp of the user through one pair, more than one pair or any combination of stimulating electrodes as determined by the brain stimulation protocol. Optionally, the stimuli generator is an isolated stimuli generator powered by a separate power unit, a constant current stimulator or V-to-I converter. Alternatively, the input/output arrangement may be connected with a constant voltage source.

The data processing arrangement includes a processing unit and a memory module. The memory module comprises of a predetermined reference data set or a set of parameters derived therefrom. Optionally, the predetermined reference data set may include EEG recordings of or data derived from EEG recordings from a plurality of persons, wherein the EEG recording is present in the form of digital electrical signals, or data that is representative thereof.

The data processing arrangement processes the detected electrical signals received from the input/output arrangement and generates the brain stimulation protocol corresponding to the received electrical signals, when in operation. Optionally, the data processing unit employs adaptive learning algorithms for processing and analysing the detected electrical signals received from the input/output arrangement. Optionally, the processed electrical signals received from the input/output arrangement are compared with one or more EEG recordings of a predetermined reference data set present in the memory module.

In an embodiment, a comparison of processed electrical signals or a set of parameters extracted from the signals with the predetermined reference data set is performed, for example, with the help of a comparator or one or more artificial intelligence algorithms or other data processing algorithms implemented in the processing unit of the data processing arrangement. Thereafter, the data processing arrangement generates an analysis of the compared electrical signals. Furthermore, the analysis optionally includes a measure of at least one: of a deviation of a parameter derived from an ideal reference signal stored in the predetermined reference data set; of a reason for such deviation from the ideal reference signal; and/or of a parameter derived following decomposition of the waveforms by individual component analysis, principal component analysis or Fourier transformation, periodogram, wavelet decomposition, wavelet transform, adaptive filters such as Wiener/Kalman filters, and other methods commonly used by those skilled in the art.

Furthermore, the data processing arrangement generates a brain stimulation protocol by implementing one or more adaptive learning algorithms or other computational algorithms after analysing the electrical signals received from the input/output arrangement. Specifically, the brain stimulation protocol may include, but is not limited to at least one of the following stimulation parameters: an amplitude, a phase, one or more frequencies with corresponding power for the brain stimuli to be generated, where these parameters are derived using one or more adaptive learning algorithms or other computational algorithms. Optionally, the brain stimulation protocol can give rise to brain stimuli in a form of a discrete signal or an arbitrary continuous waveform. Furthermore, the generated brain stimuli or the brain stimulation protocol are optionally transmitted to the input signal processing arrangement for comparison and subtraction of the generated stimulus artefacts, wherein the input signal processing arrangement is communicably coupled with the stimuli generator or with the data processing arrangement.

The brain interfacing apparatus further comprises one or more power units. The power units are electrically coupled with the input/output arrangement and the data processing arrangement and supply electrical power to the input/output arrangement and the data processing arrangement, when in operation. Optionally, the power unit may include at least one of the following sources including, but not limited to: a nickel-cadmium (NiCd), a nickel-zinc (NiZn), a nickel metal hydride (NiMH), a solid-state battery (for example, a ceramic-based battery, a glass-based battery or a sulphide-based battery) and a lithium-ion (Li-ion) or lithium-polymer (Lipo) battery, as well as a generator of power from sources like movement or solar energy, a receiver for one of wireless power transfer technologies, or a surge protected input from the mains.

In an embodiment, the brain interfacing apparatus comprises of at least two power units for providing an isolated electrical power to an input portion (comprising of units/arrangements responsible for recording or monitoring and processing of electrical signal received form the brain of the user) and an output portion (comprising of units/arrangements responsible for the generation of the brain stimuli) of the input/output arrangement, respectively.

In an embodiment, the one or more power units are operable to supply electrical power to the brain interfacing apparatus on receiving an instruction from the user via the control unit. Moreover, the user may provide the brain interfacing apparatus with an instruction to switch “ON” the electrical power supply to the brain interfacing apparatus, after wearing the headwear arrangement for initialising the operation of the brain interfacing apparatus. Optionally, the one or more power units are operable to automatically switch “ON” the electrical power supply to the brain interfacing apparatus, in a situation when the user wears the headwear arrangement of the brain interfacing apparatus.

Advantageously, the brain interfacing apparatus provides a user-friendly stimulation environment to the user for achieving desired effects of NIBS systems on the brain of the user. The desired effects may include, but are not limited to, one or more of: a cognitive enhancement of the user, an enhancement of motor control of muscles of the user, a mood enhancement of the user, an enhancement of learning of the user, an enhancement of relaxation of the user, an enhancement of concentration of the user, an alleviation of tremor afflicting the user, an alleviation of depression afflicting the user and an alleviation of epilepsy afflicting the user.

In an embodiment, the predetermined reference data set is stored in the memory module and in certain examples it could be updated iteratively in a real-time manner, when the brain interfacing apparatus is in operation.

In one embodiment, an operation of the memory module may include updating the predetermined reference data set based on electrical signals or parameters derived from these electrical signals received from the brain of the user, by storing the received electrical signals or the parameters in the memory module during the operation. In an example, the electrical signals received from the brain of the user are processed and/or modified by an input/output arrangement and then sent to the data processing arrangement. Furthermore, the data processing arrangement stores the received electrical signals in the memory module. Thereafter, the data processing arrangement compares the received electrical signals or the parameters derived from the received electrical signals with the predetermined reference data set to generate an analysis of the received electrical signals. Optionally, this may include a machine learning algorithm or other computational algorithms to update the processing used to generate a measure of a deviation of the detected electrical signal from an ideal reference signal or a set of parameters derived from the reference signal stored in the predetermined reference data set or of a reason for such deviation from the ideal reference signal.

In an embodiment, the data processing arrangement may analyse the received electrical signals in a real-time manner, so that the electrical signals are detected at the user's scalp concurrently with the brain stimuli being applied to the brain of the user.

In an example, the processed and/or modified electrical signals received from the input signal processing arrangement may be sent to the data processing arrangement for comparison with predetermined reference data set to generate an analysis of the received electrical signals, wherein at least one adaptive learning algorithm is employed to generate the analysis of the received electrical signals and at least one adaptive learning algorithm is employed to generate the brain stimulation protocol. Optionally, the brain stimulation protocol may include at least one of the following stimulation parameters: an amplitude, a signal shape as perceived when displayed on an oscilloscope screen, one or more frequencies with corresponding power and a phase difference for the brain stimuli to be applied to the brain of the user. Thereafter, the brain stimulation protocol is transmitted to the signal generator of the input/output arrangement where the signal generator generates the brain stimuli corresponding to the received brain stimulation protocol from the data processing arrangement. Subsequently, the generated brain stimuli are applied to the scalp of the user by using the at least one electrode of the plurality of electrodes. Specifically, detection, processing and analysis of electrical signals received from the brain and application of the brain stimuli to the scalp of the user are carried out concurrently or simultaneously in such a manner that there is minimal lag in the aforesaid operation.

In another embodiment, the data processing arrangement may process the electrical signals received from the input signal processing arrangement temporally alternating with the brain stimuli being applied to the user; such an approach yields potentially less cross-talk between stimuli and detected signals from the electrodes in comparison to a concurrent application of the stimuli and receiving the detected signals from the electrodes. In an example, the electrical signals received from the input signal processing arrangement are analysed by the data processing arrangement using at least one adaptive learning algorithms or other computational algorithms. Furthermore, based on the analysis, a brain stimulation protocol is generated and in accordance with the brain stimulation protocol, the brain stimuli are generated. Such recording of the received electrical signal by the input/output arrangement and application of the generated brain stimuli are carried out in an alternating manner having a small time gap in between. Furthermore, such analysis of the received electrical signal by the data processing arrangement and application of the generated brain stimuli to the user are carried out in a temporally alternate manner.

In yet another embodiment, the brain stimuli are applied to the user's scalp via the plurality of electrodes of the electrode arrangement, and to other parts of the user spatially remote from the given user's scalp, including, for example, one or more of the limbs, the spinal cord or the vagus nerve. Furthermore, the brain stimuli or stimuli to other parts of the user are generated by the stimuli generator of the input/output arrangement in accordance with the brain stimulation protocol received from the data processing arrangement. Thereafter, the generated brain stimuli are applied to the scalp and other parts of the user by the one or more of the plurality of electrodes.

Optionally, the generated brain stimuli may be applied to other body parts such as parts including, but not limited to, the neck, the spine, the heart, the chest, the abdomen, the hands, the feet, the arms and the legs which are spatially remote or located away from the scalp of the given user and here the term “electrode arrangement” includes the location of the electrode on any of the aforementioned body parts. In an example, one or more of the plurality of electrodes are in electrical contact with the neck of the user to stimulate the vagus nerve for heart rate reduction and an electrical signal is applied thereto concurrently with the brain stimuli applied to the scalp of the user.

The data processing arrangement uses at least one adaptive learning algorithm or other computational algorithms implemented as at least one of the executable software and the digital hardware (e.g. FPGA, ASIC, custom hardware Silicon chip design). Furthermore, the at least one adaptive learning algorithm may include at least one of a hardware, executable software or a digital hardware (e.g. FPGA, ASIC, custom Silicon chip design) configured to use the technology of real-time adaptation of brain stimuli in a manner that minimises the latency between signal processing and generation of the brain stimulation protocol. Moreover, the data processing arrangement, including adaptive learning algorithms, keeps track of the effects that the various brain stimulation protocols have on the brain of the user. Furthermore, such data processing arrangement is versatile enough to analyse its own actions and consequently utilise at least one of the adaptive learning algorithms or other computational algorithms to optimise the brain stimulation protocol based on the more relevant training datasets. Moreover, the training datasets may include, but are not limited to, previous action records, data from plurality of other similar systems, predetermined reference data and historical data. In an embodiment, the brain interfacing apparatus implementing the adaptive learning algorithm is configured to record and extract one or more potential target marker for neuromodulation. Optionally, the one or more potential target markers are the changes or activities caused in the brain of the user in the forms of a change of brainwaves or reduction of response to painful stimuli, wherein the changes or activities are caused in response to use of one or more drug injected to the user. In an embodiment, the one or more potential target markers are stored in databases for implementation of artificial intelligence algorithms. The brain interfacing apparatus is capable of delivering and optimising a brain stimulation protocol to induce effects similar to those induced by drugs affecting specific neuronal receptors. Beneficially, such optimal stimulation helps in inhibiting or potentiating activities similar to drugs without their side effects. In another embodiment, the brain interfacing apparatus implementing the adaptive learning algorithm is configured to stimulate or mimic the changes or activities caused in the brain of the user in the forms of a change of brainwaves based on the recorded target markers. Therefore, the use of the device and the algorithms (i) for recording and extracting potential target markers for neuromodulation; (ii) for modulating brain waves, event-related potentials or other signals to mimic the changes achieved by drugs; (iii) to enhance the effects of drugs; (iv) to reduce the unwanted side effects of drugs on the brain activity has implications for replacement of regular drugs such as opiates, or other benefits in medical conditions.

Beneficially, the adaptive learning algorithm contributes largely in achieving a more personalised and thus more effective brain stimulation for the user. Additionally, the adaptive learning algorithm or another computational algorithm continuously, in a closed loop manner, learns the patterns of response of the brain of the user to the past stimulation to better adjust the future brain stimuli for achieving optimised results. Furthermore, implementation of adaptive learning algorithms helps in enhancing the therapeutic contribution of neuromodulation devices such as the brain interfacing apparatus of the present disclosure.

In an embodiment, the adaptive learning algorithm may include, but is not limited to at least one of the machine learning algorithms which in turn include, but are not limited to: a K-nearest neighbour algorithm, a regression analysis, ensemble tree based algorithms, maximum power point tracking, a hidden Markov model, an artificial neural network, a recurrent neural network, a long short-term memory algorithm, a generative adversarial or adaptive adversarial neural networks, a convolutional neural network or a deep convolutional neural network, a reinforcement learning algorithm, random forest algorithm, an adaptive annealing algorithm, support vector machines, a recommender system, genetic algorithm, Q learning and a deep Q-learning algorithm, wherein at least one adaptive learning algorithm or another suitable computational algorithm is implemented in a closed-loop system. Furthermore, the machine learning algorithm relates to a complex source code implemented on at least one of the executable software and the digital hardware (e.g. FPGA, ASIC, custom Silicon chip design), wherein such an implementation of a machine learning algorithm is pre-trained to extract information from the input signal data or from a set of parameters derived from the input signal data in real-time with a minimal lag, or is trained in run-time by the training algorithm comparing the desired outcome with the actual outcome and adjusting the brain stimulation protocols accordingly. Moreover, the algorithm uses various rules to adjust a set of parameters, wherein the parameters are built in the algorithm to form patterns for executing a decision-making process. Optionally, in an event when a new or additional data becomes available, the algorithm when in operation, automatically adjusts the parameters to create a change in pattern by comparing the present pattern with the previous pattern.

In another embodiment, the reinforcement learning algorithm is a category of algorithms based on goal-oriented algorithms, which learn how to attain a complex objective (goal) or maximise along a particular parameter over many steps by employing a notion of a cumulative reward; for example, maximising the power and duration of high alpha activity over a prolonged period of stimulation, which acts as a cumulative reward. Moreover, the reinforcement learning algorithm learns from the rewards that it gets in response to an action performed by the system implementing reinforcement learning algorithms and adapts accordingly for maximising the cumulative rewards in the response to subsequent actions.

Furthermore, optionally, the deep Q-learning algorithm relates to the category of algorithms which includes both reinforcement learning algorithm and a neural network algorithm with multiple hidden layers for achieving an optimised output in real time manner when implemented in the closed loop system. Furthermore, the neural network algorithm relates to a series of algorithms that endeavours to recognise underlying relationships in a set of data through a process that mimics the way the human brain operates. Moreover, the neural network algorithm provides “deep learning” by way of a hierarchical arrangement of a set of parameters, wherein the parameters are built in the algorithm to form patterns for executing a decision-making process. Furthermore, in an event when a new or additional data becomes available, the algorithm when in operation, automatically adjusts the parameters to create a change in the patterns.

In an embodiment, at least one of the aforementioned adaptive learning algorithms are implemented in the closed loop system. Furthermore, the closed loop system comprises the electrode arrangement, the input/output arrangement, the data processing arrangement and the power unit. Furthermore, the input/output arrangement includes a pre-processor, the input converter, the stimuli generator and the output converter. Moreover, the data processing arrangement comprises of a processing unit and the memory module, wherein the processing unit and the memory module are communicably coupled. Furthermore, the electrode arrangement, the pre-processor, input converter, the stimuli generator, the output converter and the data processing arrangement are communicably coupled to each other either directly or indirectly. Moreover, the electrical signals generated in the brain of the user are detected by the electrode arrangement and then delivered to the processing unit through the pre-processor and input converter. Furthermore, the processing unit applies at least one of the adaptive learning algorithms to generate and deliver a brain stimulation protocol to the output converter. Furthermore, the output converter processes and transfers the processed brain stimulation protocol to the stimuli generator, wherein the stimuli generator generates the brain stimuli and delivers the generated brain stimuli to the electrode arrangement for brain stimulation of the user. Optionally, for artefact subtraction, a copy of the generated brain stimulus is also sent to the pre-processing arrangement. Moreover, the various types of signals are processed in a closed loop such that, the brain stimulation protocol is updated iteratively to reach a target electrical activity and thereby achieve a personalised and optimised brain stimulation in real-time manner.

Beneficially, the closed loop systems implementing machine learning algorithms in Non-Invasive Brain Stimulation (NIBS) systems provide real-time protocol adjustment and optimal stimulation, resulting in more personalised and efficient stimulation of the brain of the user.

Throughout the present disclosure, the term “target electrical activity”, as used herein, relates to a desired general or specific pattern of an electrical activity in the brain of the user or of parameters derived from analysis of such activity to be obtained for amelioration of symptoms associated with a particular mental health imbalance condition in humans, or another clinically relevant condition that can be alleviated with the aforementioned method. Furthermore, the target electrical activity can also be a desired electrical activity to provide or induce a specific mood, an emotion in the user's brain or another specific state of mind that can be achieved with the aforementioned method.

In an embodiment, the data processing arrangement uses at least one adaptive learning algorithm to adjust the brain stimuli iteratively, so that an electrical activity of the brain of the given user is adjusted to an approximate target electrical activity of the brain as desired. After applying the generated brain stimuli to the scalp of the user, the electrical signals from the brain of the user are detected again and analysed by the data processing arrangement in a closed loop. Optionally, analysis of the detected electrical signals includes determining changes in the detected electrical activity or in the parameters derived from the detected electrical activity of the brain subsequent to application of brain stimuli in the previous iteration. More optionally, analysing the detected electrical signals further includes determining a positive or a negative value or a set of values required for adjustment of any of the parameters of the brain stimulation protocol, in order to reach a desired target electrical activity of the brain. Such analysis by the data processing arrangement may be carried out with the help of at least one adaptive learning algorithm in a manner, such that the brain stimulation protocol to be applied may be adjusted iteratively after every brain stimuli application and detecting the effect of applied brain stimuli. Specifically, the iterative operation of adjusting the brain stimuli is performed in real-time to apply the adjusted brain stimuli to the scalp of the user to finally obtain the desired target electrical activity of the brain. In this context, real-time means that: the recording of the received electrical signal by the input/output arrangement; the processing with the data processing arrangement including the execution of adaptive learning algorithm; the adjustment of the parameters of the brain stimulation protocol; and the application of the generated brain stimuli are carried out either concurrently or in a sequential manner or in an alternating manner with the cycle being completed within a small time domain. Furthermore, optionally, the time to completion of the cycle is reduced to several milliseconds with the implementation of digital hardware for data processing and execution of adaptive learning algorithms.

Alternatively, real-time means with cycle intervals of less than 5 minutes, more optionally with cycle intervals of less than 1 minute, more optionally with cycle intervals of less than 1 second, more optionally with cycle intervals of less than 1 millisecond and yet more optionally with the assistance of the aforementioned implementations of digital hardware at cycle intervals of less than 1 microsecond.

Throughout the present disclosure, the term “feedback loop” relates to the adaptation of brain stimulation used for affecting the state of the user's brain with respect to: inter-individual structural variabilities; individual signalling dynamics and the quickly changing state of the brain in real time.

In an embodiment, the adaptation of brain stimulation automatically in real-time without involvement of any third party is referred to as the “closed loop”. Moreover, if the stimulation is not effective at changing the state of the brain towards a desired state, then the brain stimulation protocol needs to be adjusted in this feedback loop until the desired effect is achieved. Furthermore, optionally, any algorithm that processes the incoming signal or adjusts the brain stimulation protocol, which can adapt to the inter-individual and inter-state differences is defined as the adaptive learning algorithms.

In an embodiment, the brain interfacing apparatus further comprises a control unit that receives, when in operation, input from at least one of the user or a third-party device, wherein the control unit is communicably coupled with the data processing arrangement and includes a communication module for establishing a communication between the apparatus and the third-party device.

Throughout the present disclosure, the term “control unit” as used herein relates to an arrangement configured to receive an instruction from the user or the third party device via a user interface, wherein the user interface is configured to record the instruction through at least one of a button interface, a wireless interface, a touch-screen interface, a gesture interface, a microphone interface (voice detection) or a brain interfacing apparatus acting in this context to control the stimulation. Optionally, the control unit, when in operation, provides the data processing arrangement with operational parameters to personalise the brain stimulation based on the input from the user or the third-party device. Moreover, the operational parameters include at least one of an ON/OFF state, a stimulation mode, a stimulation time, an age of the user, a gender of the user, any relevant medical history and a medical condition of the user subjected to the brain stimulation or a desired mental state of the user. Optionally, the control unit arrangement uploads to the data processing arrangement a programme containing at least one of the adaptive learning algorithms designed for the optimisation of detection of brain signals specified by the programme or for the optimisation of stimulation to achieve the target electrical activity defined by the programme. Alternatively, optionally, the control unit includes the communication module for establishing a wired or wireless connection including, but not limited to a connection via the Internet, between the brain interfacing apparatus and the third-party device. Optionally, this may allow the third party device to upload a programme to the data processing arrangement via the control unit. Optionally, this may communicate the input and output signals to the third-party device, such that the third-party device is implemented as a computer, a phone (for example, a smartphone), a local server, a server arrangement (such as, an arrangement of two or more servers communicably coupled with each other), a cloud server or a quantum computer, to allow the third-party device to act as the data processing arrangement. Additionally, the control unit is configured to control an external stimulation arrangement based on the input from at least one of the user and the third-party device. Furthermore, the control unit is operable to receive electrical power from a power unit.

Throughout the present disclosure, the term “third-party device” as used herein relates to an external device communicably coupled to the control unit via the communication module, wherein the communication is realised using wired or wireless connections including, but not limited to a connection via the Internet, Bluetooth® and so forth. Optionally, the third-party device includes at least one of a smartphone, a computer (can be personal, cloud-based, distributed or a tablet computer), a smart-watch, a remote control, a medical device, a local server, a server arrangement (such as, an arrangement of two or more servers communicably coupled with each other), a cloud server and a quantum computer. More optionally, the third-party device is configured to receive a monitoring information related to electrical signals detected from the brain of the user, wherein the monitoring information includes at least one of an electroencephalogram (EEG), electric field encephalography (EFEG), Near infrared spectroscopy (NIRS), Magnetoencephalography (MEG), Electromyography (EMG), Electrocardiography (ECG), heart and/or breathing rate monitor, eye tracking and/or Functional magnetic resonance imaging (fMRI). Additionally, the third-party device is configured to control the external stimulation arrangement via the control unit. Optionally, the brain interfacing apparatus, when in operation, uses the third-party device communicably coupled with the control unit to transmit the operational parameters to the control unit, which include, but not limited to at least one of: an ON/OFF state, a stimulation mode, a stimulation time, an age of the user, a gender of the user, any relevant medical history and a medical condition of the user subjected to the brain stimulation or a desired mental state of the user. Further, optionally, the brain interfacing apparatus, when in operation, uses the third-party device to upload to the data processing arrangement via the control unit a programme containing at least one of the adaptive learning algorithms designed for the optimisation of detection of brain signals specified by the programme or for the optimisation of stimulation to achieve the target electrical activity defined by the programme.

Beneficially, the control unit and the third-party devices provide a better interaction with the user through a user-friendly interface. Optionally, the control unit enables the third-party device to execute a customised adaptive learning algorithm instead of the data processing unit, which can be beneficial where the processing power required for the execution of the adaptive learning algorithm exceeds that of a data processing unit. Moreover, the use of third-party devices enables the user to customise operational parameters of the apparatus for generating a customised brain stimulation protocol. Advantageously, the brain interfacing apparatus also provides an open platform for scientists and doctors to explore the functional aspects of the human brain in a much more detailed and in a real-time manner (as aforementioned) with the help of monitoring information such as the electroencephalogram (EEG), electric field encephalography (EFEG), Near infrared spectroscopy (NIRS), Magnetoencephalography (MEG), Electromyography (EMG), Electrocardiography (ECG), eye tracking and/or functional magnetic resonance imaging (fMRI).

In an embodiment, the apparatus further comprises an external stimulation arrangement for providing at least one of: a visual stimulation, audio stimulation and/or a virtual reality stimulation to the user's brain, wherein the external stimulation arrangement is communicably coupled with the control unit. Optionally, the external stimulation arrangement communicates with the data processing arrangement directly or via the control unit. More optionally, at least one of: a visual stimulation, an audio stimulation and/or a virtual reality stimulation to the user's brain, provided by the external stimulation arrangement is in synchronisation with the brain stimuli applied to the brain of the user.

In an embodiment, the parameters of at least one of: a visual stimulation, audio stimulation and/or a virtual reality stimulation become a part of a brain stimulation protocol optimised by the control unit.

Throughout the present disclosure, the term “external stimulation arrangement” as used herein relates to a detachably coupled external device used for audio-visual or virtual-reality stimulation using at least one of a virtual reality device, a display device, glasses, headphones, earphones, a speaker, a therapeutic massager, electrodes placed elsewhere on the body and/or a smart-lens (such as a Google Lens®). Moreover, the external stimulation arrangement is configured to receive electrical power from one or more power unit.

In an example, the external stimulation arrangement provides audio-visual stimulation for relaxing the user and bringing down the stress level when operated in synchronisation with the brain stimuli. Advantageously, the external stimulation arrangement provides isolation to the user by reducing the unwanted light coming to the eyes of the user and noise coming to the ears of the user. Such an isolation helps the user to further reduce unwanted brain activity, resulting in enhanced effectiveness of the brain stimulation protocols.

In exemplary operation, the control unit is implemented as a microcontroller associated with the stimuli generator and/or the external stimulation arrangement. Furthermore, the third-party device is implemented as a laptop computer (for example, a MacBook™ laptop computer), such that the microcontroller is communicably coupled with the laptop computer via a cloud-based platform. The laptop computer processes the operational parameters associated with brain stimulation to be provided to a user and subsequently, transmits the operational parameters to the microcontroller associated with the external stimulation arrangement. Furthermore, the laptop computer transmits the operational parameters to the microcontroller in real-time. In such an example, the external stimulation arrangement comprises a Light Emitting Diode (referred to as “LED” hereinafter) or alternatively, an assembly of LEDs and the communication module comprises a WiFi chip, such that, the LED is connected with the microcontroller and the microcontroller is communicably coupled with the laptop via the cloud-based platform. Furthermore, the laptop computer controls the brain stimuli delivered using the LED, such as, by regulating a frequency, pulse-width and/or brightness of light emitted by the LED. Furthermore, a plurality of electrodes comprises a pair of electrodes arranged on the scalp of the user corresponding to a location of an occipital lobe (such as, at O1 and O2 locations, in accordance with 10-20 system of EEG positioning) and a reference electrode and bias electrodes are arranged on temples of the user (such as, at T3 and T4 locations respectively). The plurality of electrodes record activity of a visual cortex of the user, such that the activity reflects a perception of the user associated with visual stimuli delivered by LED. The plurality of electrodes is communicably coupled to the input/output arrangement that can be implemented using an OpenBCI Cyton PCB. The input/output arrangement has a programmable gain analog-to-digital converter to amplify and convert analog signals detected across each of the plurality of electrodes into digital data. Furthermore, the input/output arrangement is communicably coupled with the third-party device implemented as the laptop computer and wirelessly transmits the digital data to the third-party device. In this manner, the third-party device receives the information from the brain via the input/output arrangement and acts as the data processing arrangement, to generate and optimise the brain stimulation protocol delivered through the aforementioned LEDs.

In another exemplary operation, the control unit is implemented as a microcontroller associated with the stimuli generator and/or the external stimulation arrangement. Furthermore, the third-party device is implemented as a smartphone. In such an example, an application software (or an “app”) is installed on the smartphone, such that the smartphone (or a user associated with the smartphone) transmits and receives operational parameters associated with brain stimulation to be provided to a user, to the headwear arrangement and/or the external stimulation arrangement via the app. In one example, such operational parameters correspond to one or more operating modes of the brain interfacing apparatus. In another example, the smartphone (or the user associated with the smartphone) can measure at least one of: a current transmitted to the plurality of electrodes for providing the brain stimulation, a voltage of the current transmitted for providing the brain stimulation (such as, a voltage required for transmitting constant current to a plurality of electrodes associated with the electrode arrangement) and an impedance at the plurality of electrodes of the electrode arrangement (such as, to determine that the plurality of electrodes is properly arranged on the scalp of the user). In yet another example, the plurality of electrodes can be arranged over a mastoid process of a temporal bone of the user, to target cranial nerves and deeper areas of the brain of the user. For example, in order to create an input for the input/output arrangement, the plurality of the electrodes (configured to record brain activity of the user) is arranged over frontal parts of the brain. Furthermore, the plurality of electrodes is connected to the input/output arrangement with an amplifier and a digital-to-analog converter that can be implemented through a modification of the OpenBCI Cyton PCB, such that, the modified OpenBCI Cyton PCB can be communicably coupled via Internet with the data processing arrangement via the cloud-based platform as well as the app installed in the third-party device.

In an embodiment, at least one of the stimuli generator and another part of the hardware arrangement include a safety arrangement, wherein the safety arrangement disables the delivery of the brain stimuli to the electrode arrangement, in an event of an electrical malfunction of the apparatus or a request from the user to cease brain stimulation. Furthermore, the safety arrangement includes at least one of a protective relay, an over-current sensor, an over-voltage sensor, a frequency sensor, a sensor of excessive muscle/movement activity (“discomfort” sensor) and an emergency “kill” switch. Furthermore, the safety arrangement is communicably coupled to the control unit via the data processing arrangement, which in turn is also coupled to the third-party device with a user-friendly interface for aborting the stimulation/recording. Moreover, the safety arrangement, when in operation, receives data related to at least one of the current and voltage at the plurality of electrodes, from at least one of the over-current sensor and over-voltage sensor. Furthermore, in one of the implementations of the safety arrangement, when in operation, it determines an event of the electrical malfunction by comparing the data related to at least one of the current and voltage at the plurality of electrodes with a pre-determined reference data including a reference data related to at least one of the current and voltage at the plurality of electrodes. Furthermore, optionally, the safety arrangement is also implemented in the data processing arrangement, the electrode arrangement, the one or more power unit and the external stimulation arrangement.

Throughout the present disclosure, the term “electrical malfunction” as used herein relates to the undesirable amount of electrical current and/or electrical voltage occurring in the brain interfacing apparatus, wherein such undesirable amount of electrical current and/or electrical voltage may harm the user and/or the apparatus. Furthermore, in an event of the electrical malfunction, the safety arrangement is configured to cut-off the electrical power supply to the apparatus from the one or more power unit via the protective relay.

Beneficially, the safety arrangement provides enhanced protection from any damage to the user in real-time manner, resulting in risk-free usage of the brain interacting apparatus without any expert assistance. Moreover, the brain interfacing apparatus is designed in its external and internal component parts, and also in its manner of operation, in such a way that any occurrence of harm for the user is avoided.

The present disclosure also relates to the method as described above. Various embodiments and variants disclosed above apply mutatis mutandis to the method.

Optionally, the method includes using the data processing arrangement for updating the predetermined reference data set iteratively in a real-time manner and storing the updated predetermined reference data set in the memory module. “Real-time” is to be understood as described in the present disclosure and need not be merely temporally continuous.

Optionally, the method includes using the data processing arrangement to analyse the received electrical signals in a real-time manner, so that the electrical signals are detected at the user's scalp concurrently with the brain stimuli being applied to the user.

Optionally, the method includes using the data processing arrangement for analysing the electrical signals received from the signal processing arrangement temporally alternately with the brain stimuli being applied to the user.

Optionally, the method includes using the plurality of electrodes of the electrode arrangement to apply the brain stimuli to the user's scalp, and to other parts of the user spatially remote from the user's scalp.

Optionally, the method includes using at least one adaptive learning algorithm or another computational algorithm, implemented within the data processing arrangement as at least as one of executable software and digital hardware (e.g. FPGA, ASIC, custom chip design).

Optionally, the adaptive learning algorithm includes, but is not limited to at least one of the machine learning algorithms which in turn include, but are not limited to: a K-nearest neighbour algorithm, a regression analysis, ensemble tree based algorithms, maximum power point tracking, a hidden Markov model, an artificial neural network, a recurrent neural network, a long short-term memory algorithm, a generative adversarial or adaptive adversarial neural networks, a convolutional neural network or a deep convolutional neural network, a reinforcement learning algorithm, random forest algorithm, an adaptive annealing algorithm, support vector machines, a recommender system, genetic algorithm, Q learning and a deep Q-learning algorithm, wherein at least one adaptive learning algorithm or another suitable computational algorithm is implemented in a closed-loop system.

Optionally, the method includes programming the data processing arrangement to use, but not limited to at least one adaptive learning algorithm to adjust iteratively the brain stimulation protocol, so that electrical activity of the brain of the user is adjusted to an approximate target electrical activity of the brain as desired.

Optionally, the method includes using a control unit to receive input from at least one of the user or a third party device, wherein the control unit is communicably coupled with the data processing arrangement and includes a communication module for establishing a communication between the apparatus and the third party device.

Optionally, the method includes using an external stimulation arrangement for providing at least one of: a visual stimulation, audio stimulation and/or a virtual reality stimulation to the brain of the user, wherein the external stimulation arrangement is communicably coupled with the control unit. In one example, the external stimulation arrangement is used for providing the visual stimulation as a transient response to the eyes of the user.

Optionally, the method includes using a safety arrangement to disable application of the brain stimuli to the plurality of electrodes, in an event of the device malfunction, wherein the safety arrangement is communicably coupled with the input/output arrangement.

In an embodiment, the present disclosure provides a computer programme product comprising a non-transitory computer-readable storage medium having computer-readable instructions stored thereon, the computer-readable instructions being executable by a computerised device comprising processing hardware to execute a method of using a brain interfacing apparatus that provides, when in operation, brain activity monitoring and stimulation of the brain of the user.

### DETAILED DESCRIPTION OF THE DRAWINGS

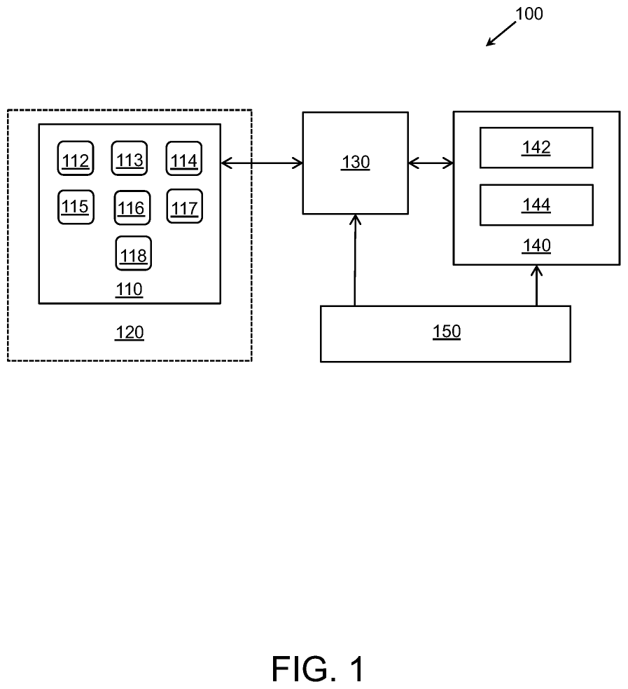

Referring FIG. 1, there is shown a block diagram of a brain interfacing apparatus 100 for brain activity monitoring and stimulation of the brain of the user, in accordance with an embodiment of the present disclosure. As shown, the brain interfacing apparatus 100 for brain activity monitoring and stimulation of the brain of the user comprises a headwear arrangement 120, a data processing arrangement 140, an input/output arrangement 130 and one or more power units 150. Furthermore, the headwear arrangement 120 comprises of an electrode arrangement 110 including a plurality of electrodes 112 to 118, wherein the plurality of electrodes is arranged in a manner to make contact with the scalp of the user, for detecting the brain activity. Moreover, the electrode arrangement 110 is communicably coupled to the input/output arrangement 130, wherein the input/output arrangement 130, when in operation, receives the detected signals and delivers the brain stimuli to the at least one of the plurality of electrodes 112 to 118. Furthermore, the input/output arrangement 130 contains an optional input signal pre-processing arrangement (not shown), which can include an optional amplifier (not shown); an artefact filter (not shown); an input converter (not shown); an output converter (not shown) and stimuli generator (not shown). Furthermore, the input/output arrangement 130 is communicably coupled with the data processing arrangement 140. Moreover, the data processing arrangement 140 comprises a memory module 142 and a processing unit 144. The one or more power unit 150, when in operation, provides electrical power to the input/output arrangement 130 and the data processing arrangement 140.

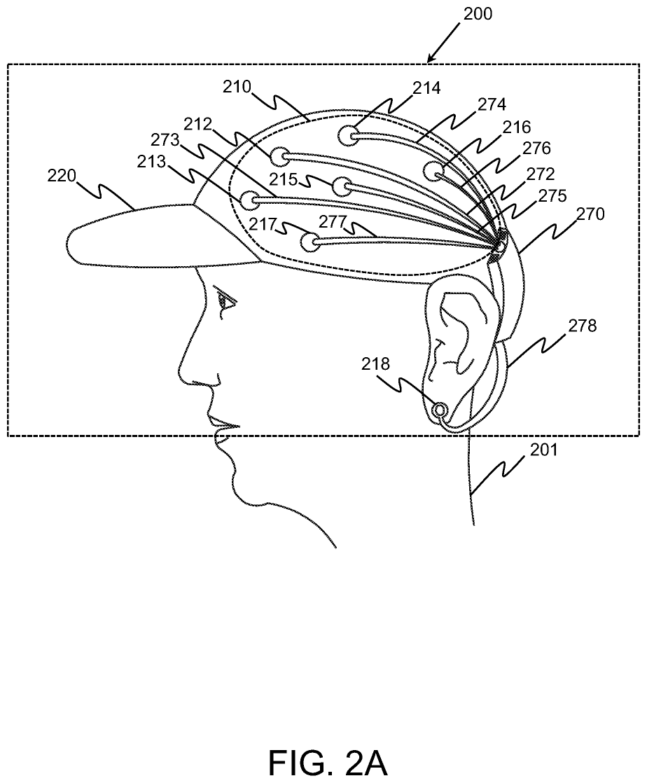

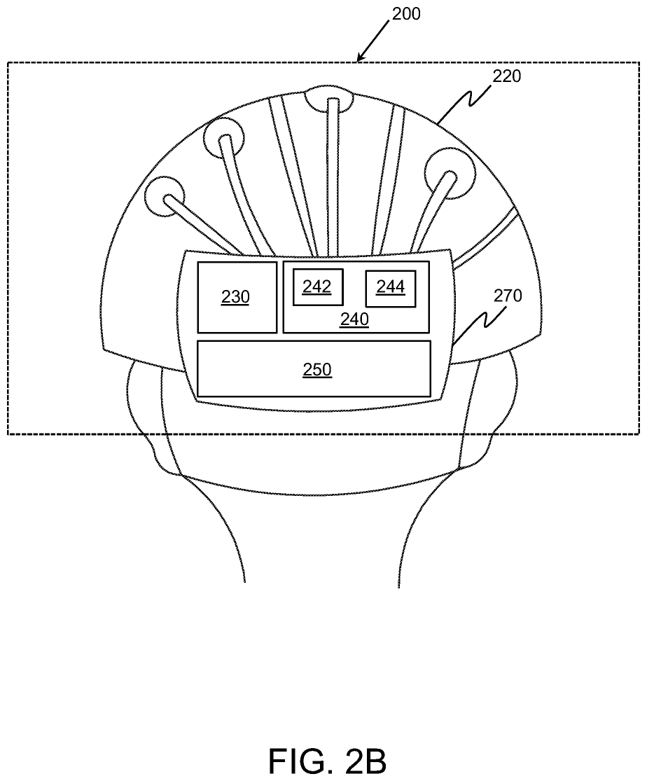

Referring FIG. 2A, there is shown an exemplary implementation of the brain interfacing apparatus 200 (such as the brain interfacing apparatus 100 of FIG. 1) positioned on the head of the user 201, in accordance with an embodiment of the present disclosure. Specifically, the exemplary implementation is a side-view of the user 201 wearing the brain interfacing apparatus 200. The brain interfacing apparatus 200 comprises a headwear arrangement 220 (such as the headwear arrangement 120 of FIG. 1) and an assembly unit 270, wherein the headwear arrangement 220 is implemented using a sports cap in this example. Moreover, the headwear arrangement 220 comprises an electrode arrangement 210 (such as the electrode arrangement 110 of FIG. 1), wherein the electrode arrangement 210 comprises of plurality of electrodes 212 to 218 (such as the plurality of electrodes 112-118 of FIG. 1). Furthermore, the plurality of electrodes 212 to 218 are connected to the assembly unit 270 through a plurality of connecting wires 272 to 278, respectively. Specifically, one of the electrode 218 of the plurality of the electrodes 212 to 218 is a reference electrode connected to a non-scalp portion of the head of the user 201.

Referring FIG. 2B, there is shown the same exemplary implementation of the brain interfacing apparatus 200 placed on the head of the user 201, in accordance with an embodiment of the present disclosure. Specifically, the exemplary implementation is a back-view of the user 201 wearing the brain interfacing apparatus 200 comprising of the headwear arrangement 220 and an assembly unit 270. Further, the assembly unit 270 comprises of a input/output arrangement 230 (such as the input/output arrangement 130 of FIG. 1), a data processing arrangement 240 (such as the data processing arrangement 140 of FIG. 1) and one or more power unit 250 (such as the one or more power unit 150 of FIG. 1). Moreover, the data processing arrangement 240 comprises a memory module 242 (such as the memory module 142 of FIG. 1) and a processing unit 244 (such as the processing unit 144 of FIG. 1).

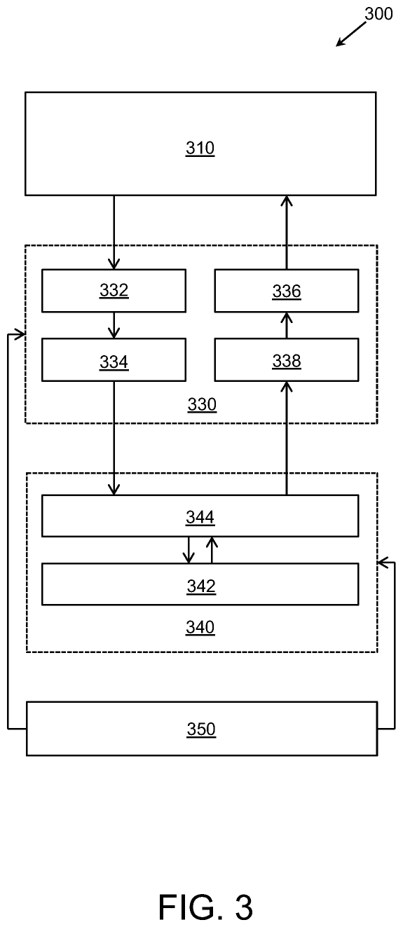

Referring FIG. 3, there is illustrated a brain interfacing apparatus (such as the brain interfacing apparatus 100 of FIG. 1) working as a closed loop system 300. The closed loop system 300, when in operation, implements at least one adaptive learning algorithm or another computational algorithm, in accordance with an embodiment of the present disclosure. The closed loop system 300 comprises an electrode arrangement 310 (such as the electrode arrangement 110 of FIG. 1), the input/output arrangement 330 (such as the input/output arrangement 130 of FIG. 1), the data processing arrangement 340 (such as the data processing arrangement 140 of FIG. 1) and the one or more power unit 350 (such as the one or more power unit 150 of FIG. 1). Furthermore, the input/output arrangement 330 includes a pre-processor 332, an input converter 334, a stimuli generator 336 and an output converter 338. The data processing arrangement 340 comprises a processing unit 344 (such as the processing unit 144 of FIG. 1) and a memory module 342 (such as the memory module 142 of FIG. 1), wherein the processing unit 344 and the memory module 342 are communicably coupled. The electrode arrangement 310, the pre-processor 332, input converter 334, the stimuli generator 336, the output converter 338 and the data processing arrangement 340 are communicably coupled in the manner shown. The electrical signals generated within the brain of the user are detected by the electrode arrangement 310 and then delivered to the processing unit 344 through the pre-processor 332 and input converter 334. The processing unit 344 applies the at least one adaptive learning algorithm or another computational algorithm to generate and deliver a brain stimulation protocol to the output converter 338. Further, the output converter 338 processes and transfers the processed brain stimulation protocol to the stimuli generator 336, wherein the stimuli generator 336 generates the brain stimuli and delivers the generated brain stimuli to the electrode arrangement 310 for brain stimulation of the user. Further, the one or more power unit 350, when in operation, supplies electrical power to the input/output arrangement 330 and the data processing arrangement 340.

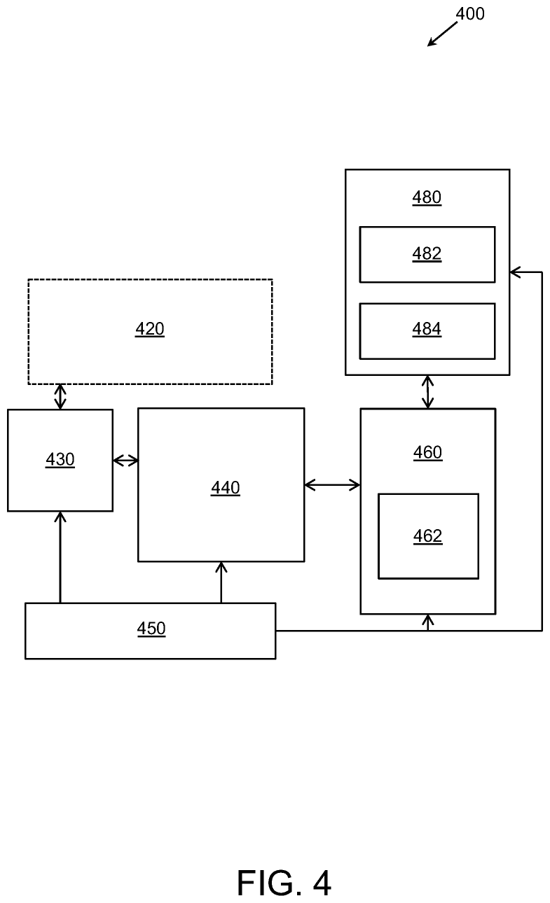

Referring FIG. 4, there is shown a block diagram of an exemplary implementation of the brain interfacing apparatus 400 (such as the brain interfacing apparatus 100 of FIG. 1) comprising the data processing arrangement 440 (such as the data processing arrangement 140 of FIG. 1), the headwear arrangement 420 (such as the headwear arrangement 120 of FIG. 1), the input/output arrangement 430 (such as the input/output arrangement 130 of FIG. 1), the data processing arrangement 440 (such as the data processing arrangement 140 of FIG. 1), the one or more power unit 450 (such as the one or more power unit 150 of FIG. 1), a control unit 460 and an external stimulation arrangement 480, in accordance with an embodiment of the present disclosure. Further, the data processing arrangement 440 of the brain interfacing apparatus 400 is communicably coupled to the control unit 460. The control unit 460 further comprises a communication module 462. Furthermore, the control unit 460 is communicably coupled to the external stimulation arrangement 480 comprising in this example an audio stimulation arrangement 482 and a virtual reality stimulation arrangement 484. Additionally, the one or more power unit 450 of the brain interfacing apparatus 400, when in operation, supplies electrical power to the data processing arrangement 440 (such as the data processing arrangement 140 of FIG. 1), and may also optionally supply electrical power to the control unit 460 and the external stimulation arrangement 480.

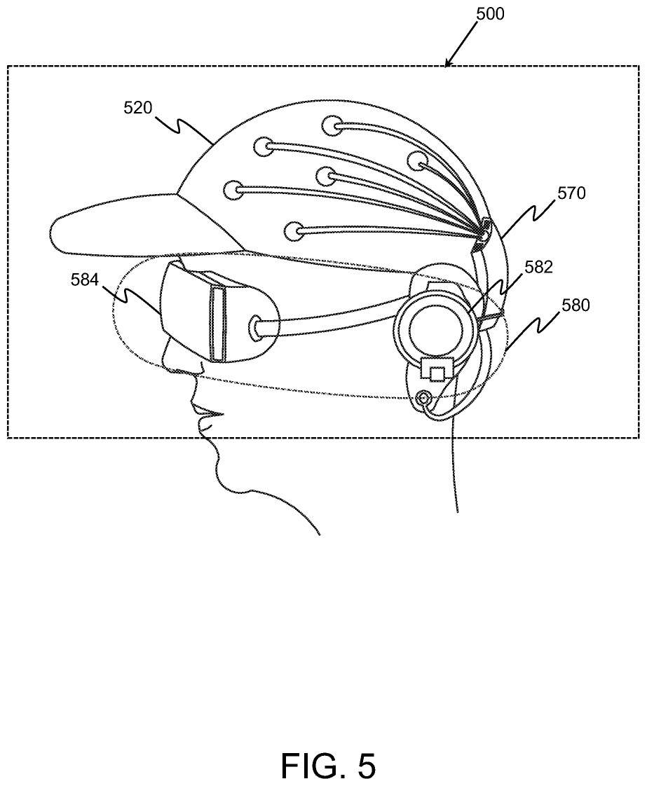

Referring FIG. 5, there is shown an exemplary implementation of the brain interfacing apparatus 500 (such as the apparatus 400 of FIG. 4) comprising an assembly unit 570 (such as the assembly unit 270 of FIGS. 2A and 2B), a headwear arrangement 520 (such as the headwear arrangement 120 of FIG. 1) and an external stimulation arrangement 580 (such as the external stimulation arrangement 480 of FIG. 4), in accordance with an embodiment of the present disclosure. The external stimulation arrangement 580 comprises in this example the audio stimulation arrangement 582 (such as the audio stimulation arrangement 482 of FIG. 4), and the virtual reality stimulation arrangement 584 (such as the virtual reality stimulation arrangement 484 of FIG. 4). Further, the external stimulation arrangement 580 is communicably coupled to the control unit (not shown). Furthermore, the assembly unit 570 (such as the assembly unit 270 of FIGS. 2A and 2B) includes the control unit (not shown) and the one or more power unit (not shown). Moreover, the one or more power unit, when in operation, may also optionally supply electrical power to the external stimulation arrangement 580.

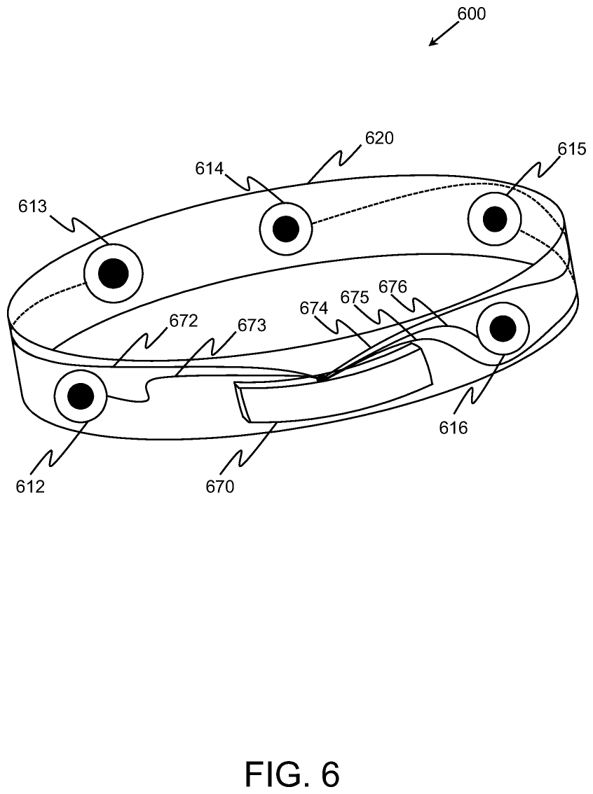

Referring FIG. 6, there is shown an exemplary implementation of the brain interfacing apparatus 600 (such as the apparatus 100 of FIG. 1) with a band type headwear arrangement 620, in accordance with an embodiment of the present disclosure. The brain interfacing apparatus 600 further comprises an assembly unit 670 (such as the assembly unit 270 of FIGS. 2A and 2B), Further, the headwear arrangement 620 (such as the headwear arrangement 120 of FIG. 1) comprises an electrode arrangement (such as the electrode arrangement 110 of FIG. 1) including the plurality of electrodes 612 to 616 (such as the plurality of electrodes 112 to 118 of FIG. 1), wherein the plurality of electrodes 612 to 616 are connected to the assembly unit 670 through the plurality of connecting wires 672 to 676 (such as the plurality of connecting wires 272 to 278 of FIGS. 2A and 2B).

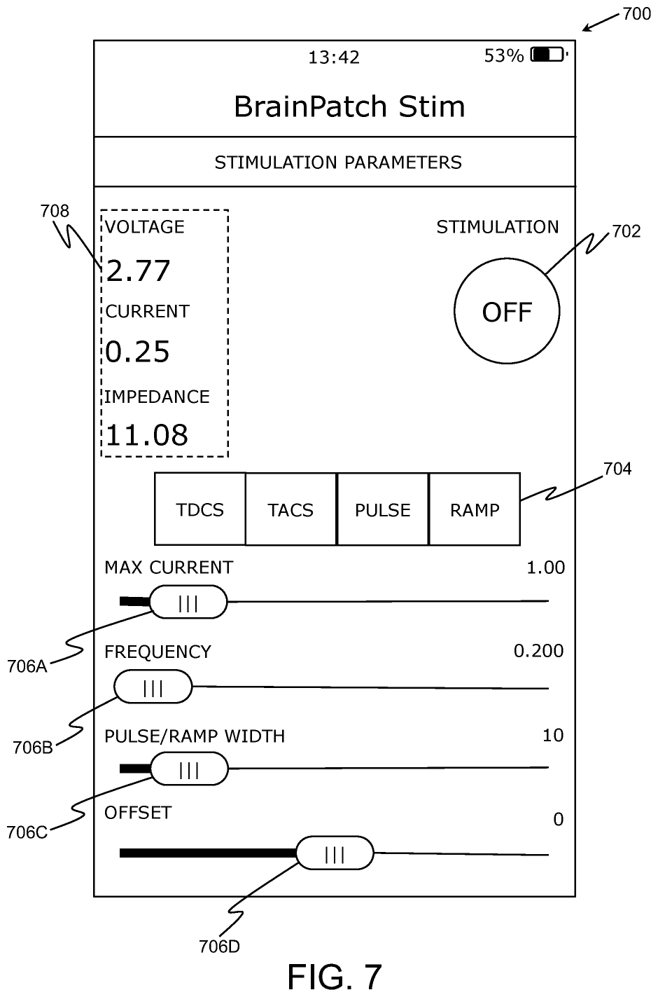

Referring to FIG. 7, there is shown an exemplary user interface 700 for receiving instruction from a user or for displaying a personalized brain stimulation applied to the user, in accordance with an embodiment of the present disclosure. As shown, the user interface 700 can be used by the user to provide instructions, such as, associated with an ON/OFF state using a button 702, a stimulation mode from amongst tDCS, tACS, pulse or ramp using corresponding buttons 704 and so forth. The user interface 700 also allows the user to regulate check the current transmitted to a plurality of electrodes, a frequency of tACS, pulses, or light emitted by an LED associated with an external stimulation arrangement, a pulse/ramp width and/or offset by using corresponding sliders 706A-D. Alternatively, the user can check the current transmitted to the plurality of electrodes, the frequency of tACS, pulses, or light emitted by the LED associated with the external stimulation arrangement, the pulse/ramp width and/or offset that are displayed using corresponding sliders 706A-D, such that the corresponding sliders 706A-D automatically change a position thereof on the user interface 700 based on updated values determined by a stimulation optimisation algorithm. Moreover, the user interface 700 displays various stimulation parameters, such as, voltage, current and impedance applied to the plurality of electrodes for providing the brain stimulation via an output area 708 of the user interface 700.

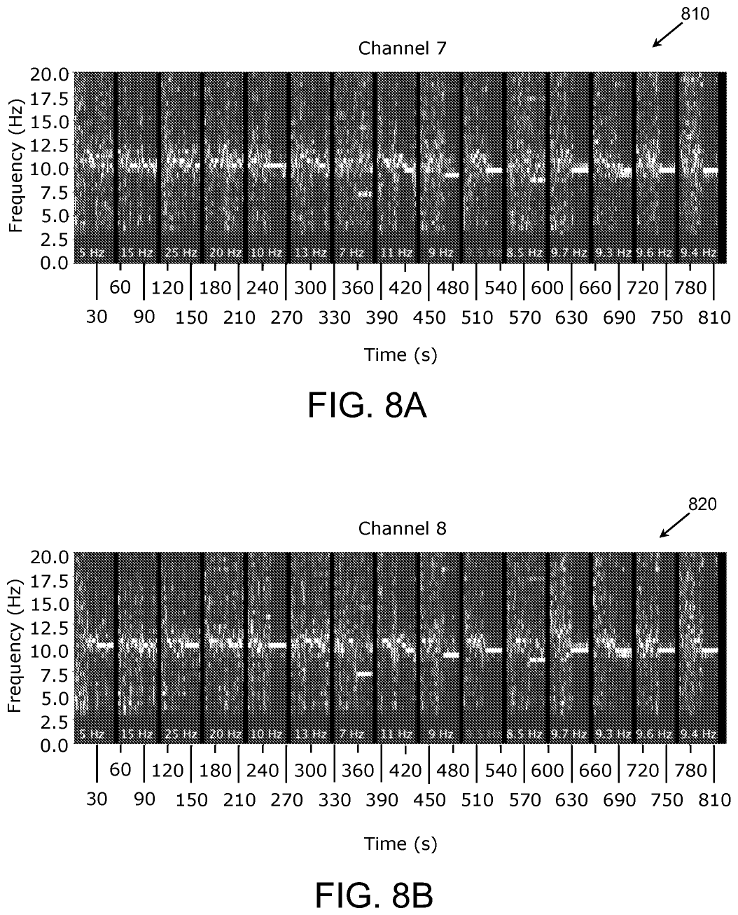

Referring to FIGS. 8A-B, there are shown spectrograms 810 and 820 of signals detected from O1 (channel 7 810 and channel 8 820 respectively) region of a brain of a user, in response to various stimulation frequencies 810-820 used for determination of an optimal stimulation frequency for a user, in accordance with an embodiment of the present disclosure. The stimulation frequencies 810-820 are optimised for a maximum change in power of brain signal with frequency corresponding to a stimulation frequency with an LED light by an adaptive maximum power point tracking algorithm. The stimulation frequencies 810-820 are applied to the LEDs for 25 seconds, each after an inactive baseline period of 25 seconds. The adaptive maximum power point tracking algorithm determines a next change in stimulation frequency, based on a position of a local maxima. Furthermore, an amplitude of such a change in stimulation frequency is varied to allow precise determination of the optimal stimulation frequency. Correspondingly, the amplitude of the applied stimulation frequency is changed until the optimal stimulation frequency is determined with a precision less than +/−0.1 Hz.

As shown, the adaptive maximum power point tracking algorithm determines first a stimulation frequency band of around 10 Hz to become prominent (depicted by a white line in spectrogram 810 of FIG. 8A along a right part of the 10 Hz column) and frequencies around 10 Hz are tested to narrow down to the optimal stimulation frequency. Moreover, when the adaptive maximum power point tracking algorithm employs various stimulation frequencies around 9.5 Hz, no further increase is identified by the power point tracking algorithm. Consequently, the adaptive maximum power point tracking algorithm determines the optimal stimulation frequency to be 9.5 Hz for the given user.

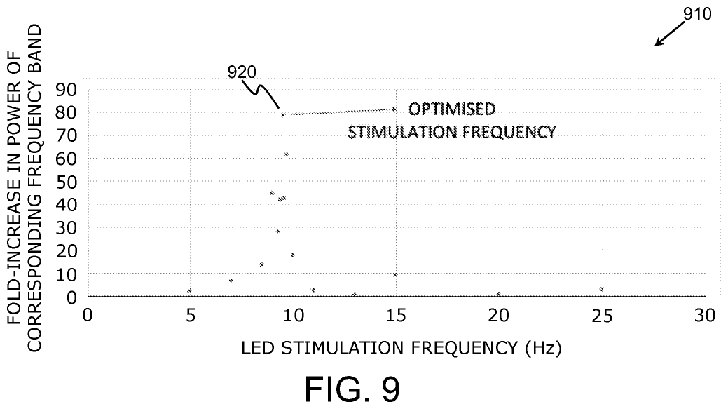

Referring to FIG. 9, there is shown a graph 910 illustrating a non-linear relationship between stimulation frequency delivered by LEDs and response power of brain signal with frequency corresponding to stimulation frequency with LED light, in accordance with an embodiment of the present disclosure. The non-linear relationship between the stimulation frequency applied to the LEDs and the response power is determined using an adaptive maximum power point tracking algorithm. The adaptive maximum power point tracking algorithm determines local maxima through application of various stimulation frequencies and narrowing down to the optimal stimulation frequency. Subsequently, the adaptive maximum power point tracking algorithm determines the local maxima to be around 10 Hz (indicated at 920 in graph 910). Furthermore, the adaptive maximum power point tracking algorithm attempts to determine the optimal stimulation frequency around the local maxima through application of various stimulation frequencies near 10 Hz. It will be appreciated that such a technique of determination of the optimal stimulation frequency of a flashing LED using the adaptive maximum power point tracking algorithm can be employed, for example, in Brain Computer Interface-related (or BCI-related) applications that rely on Steady State Visually Evoked Potentials. The optimal stimulation frequency in such BCI-related applications can be used for generating a reliable response to flickering visual stimulations, such as, to more accurately and quickly guide equipment that a user is attempting to control with their brain.

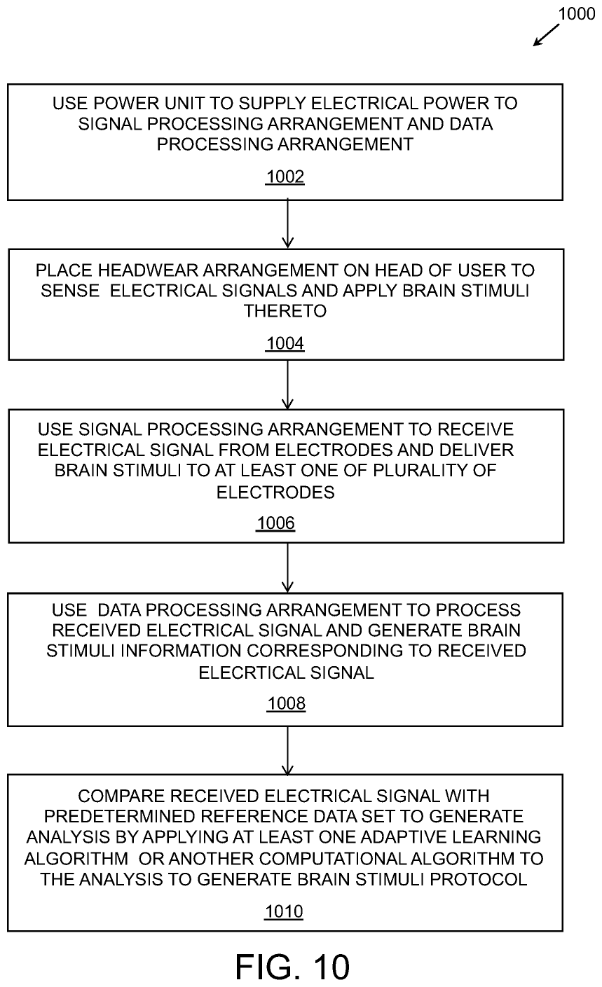

Referring to FIG. 10, illustrated are steps of a method 1000 for brain activity monitoring and stimulation of the brain of the user by using a brain interfacing apparatus (such as the apparatus 100 of FIG. 1), in accordance with an embodiment of the present disclosure. The method initiates at a step 1002, at the step 1002, one or more power units (such as the one or more power unit 140 of FIG. 1) are used to supply electrical power to an input/output arrangement and a data processing arrangement. At a step 1004 a headwear arrangement (such as the headwear arrangement 120 of FIG. 1) is placed on the head of the user to detect electrical signals and apply a brain stimuli thereto. At a step 1006, the input/output arrangement (such as the input/output arrangement 130 of FIG. 1) is used to receive electrical signal from a plurality of electrodes (such as the plurality of electrodes 112 to 118 of FIG. 1) and deliver the brain stimuli to at least one, to a pair or to any combination of the plurality of electrodes. At a step 1008, the data processing arrangement (such as the data processing arrangement 140 of FIG. 1) is used to process the received electrical signal and generate a brain stimulation protocol corresponding to received electrical signal. Optionally, the received electrical signal is processed by applying at least one of an adaptive learning algorithms or another computational algorithm to generate the brain stimulation protocol corresponding to the received electrical signal. At a step 1010, the data processing arrangement compares the received electrical signal with a predetermined reference data set to generate an analysis by applying at least one of the adaptive learning algorithms or another computational algorithm to generate the brain stimulation protocol. The method 1000 ends at the step 1010 if a predetermined goal of the stimulation or a predetermined stopping point is reached, otherwise steps 1004 to 1010 are repeated automatically in an iterative manner until a predetermined goal of the stimulation or a predetermined stopping point is reached. Additionally, the process from 1004 to 1010 may function iteratively based on the instructions received from the data processing arrangement (such as the data processing arrangement 140 of FIG. 1).

The steps 1002 to 1010 are only illustrative and other alternatives can also be provided where one or more steps are added, one or more steps are removed, or one or more steps are provided in a different sequence without departing from the scope of the claims herein.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural where appropriate.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

1-23. (canceled)

24. A brain interfacing apparatus that provides, when in operation, brain activity monitoring and stimulation of the brain of a user, wherein the apparatus comprises:
(i) a headwear arrangement to be placed or positioned on a head of the user wherein the headwear arrangement comprises an electrode arrangement including a plurality of electrodes that makes electrical contact with a scalp of the user, when in operation, to detect electrical signals therefrom and to apply a brain stimuli thereto;
(ii) an input/output arrangement that receives electrical signals from at least one of the plurality of electrodes and delivers the brain stimuli using a brain stimulation protocol to the at least one of the plurality of electrodes, when in operation;
(iii) a data processing arrangement that processes the detected electrical signals received from the input signal processing arrangement and generates the brain stimulation protocol corresponding to the received electrical signals, when in operation, wherein the data processing arrangement includes a memory module; and
(iv) one or more power units that supply electrical power to the input/output arrangement and the data processing arrangement,
wherein the data processing arrangement compares the received electrical signals with a predetermined reference data set to generate an analysis of the received electrical signals and applies at least one adaptive learning algorithm or another computational algorithm to the process of analysing and generating the brain stimulation protocol.

25. The brain interfacing apparatus of claim 24, wherein the predetermined reference data set is stored in the memory module and updated iteratively in a real-time manner, when the brain interfacing apparatus is in operation.

26. The brain interfacing apparatus of claim 24, wherein the data processing arrangement analyses the received electrical signals and applies the brain stimulation protocol in a real-time manner, so that the electrical signals are detected at the user's scalp concurrently with the brain stimuli being applied to the user.

27. The brain interfacing apparatus of claim 24, wherein the data processing arrangement analyses the electrical signals received from the input signal processing arrangement temporally with the brain stimuli being applied to the user.

28. The brain interfacing apparatus of claim 24, wherein the stimuli are also applied to other parts of the user spatially remote from the given user's scalp.

29. The brain interfacing apparatus of claim 24, wherein the data processing arrangement uses, but not limited to the at least one adaptive learning algorithm or another computational algorithm implemented at least as: executable software, digital hardware (e.g. FPGA, ASIC, custom chip design).

30. The brain interfacing apparatus of claim 24, wherein the at least one adaptive learning algorithm includes, but not limited to at least one of the machine learning algorithms: a K-nearest neighbour algorithm, a regression analysis, ensemble tree based algorithms, maximum power point tracking, an artificial neural network, a deep convolutional neural network, a recurrent neural network, a reinforcement learning algorithm, random forest algorithm, a recommender system, genetic algorithm, Q-learning and a deep Q-learning algorithm, wherein the at least one of those or another computational algorithm is implemented in a closed-loop system.

31. The brain interfacing apparatus of claim 24, wherein the data processing arrangement uses, but not limited to the at least one adaptive learning algorithm or another computational algorithm to adjust iteratively the brain stimulation protocol, so that the electrical activity of the brain of the user is modulated to an approximate target as desired.

32. The brain interfacing apparatus of claim 24, wherein the apparatus further comprises a control unit that receives, when in operation, input from at least one of the user or a third party device, wherein the control unit is communicably coupled with the data processing arrangement and includes a communication module for establishing a communication between the apparatus and the third party device.

33. The brain interfacing apparatus of claim 24, wherein the apparatus further comprises an external stimulation arrangement for providing at least one of: a visual stimulation, an audio stimulation and/or a virtual reality stimulation to the user's brain, wherein the external stimulation arrangement is communicably coupled with the control unit.

34. The brain interfacing apparatus of claim 24, wherein the input/output arrangement includes a safety arrangement, wherein the safety arrangement disables applying any brain stimuli to the electrode arrangement and recording from the electrode arrangement, in an event of a device malfunction of the apparatus.

35. A method for using a brain interfacing apparatus that provides, when in operation, brain activity monitoring and stimulation of the brain of a user, wherein the method includes:
(i) using one or more power unit to supply electrical power to an input/output arrangement and a data processing arrangement;
(ii) placing or positioning a headwear arrangement on a head of the user, wherein the headwear arrangement comprises an electrode arrangement including a plurality of electrodes that makes electrical contact with a scalp of the user, when in operation, to detect electrical signals therefrom and to apply a brain stimuli thereto;
(iii) using the input/output arrangement to receive electrical signals from at least one of the plurality of electrodes and to deliver the brain stimuli using a brain stimulation protocol to the at least one of the plurality of electrodes;
(iv) using the data processing arrangement to process the detected electrical signals received from the input/output arrangement and to generate the brain stimulation protocol corresponding to the received electrical signals, wherein the data processing arrangement includes a memory module; and
(v) comparing the received electrical signals and a predetermined reference data set for generating an analysis and applying at least one adaptive learning algorithm or another computational algorithm to the analysis for generating the brain stimulation protocol.

36. The method of claim 35, wherein the method includes using the data processing arrangement for updating the predetermined reference data set iteratively in a real-time manner and storing the updated predetermined reference data set in the memory module.

37. The method of claim 35, wherein the method includes using the data processing arrangement to analyse the received electrical signals in a real-time manner, so that the electrical signals are detected at the user's scalp concurrently with the brain stimuli being applied to the user.

38. The method of claim 35, wherein the method includes using the data processing arrangement for analysing the electrical signals received from the input/output arrangement temporally with the brain stimuli being applied to the user.

39. The method of claim 35, wherein the method includes using at least one of the plurality of electrodes of the electrode arrangement to apply the brain stimuli to the user's scalp, and to other parts of the user spatially remote from the user's scalp.

40. The method of claim 35, wherein the method includes arranging for the data processing arrangement to use, but not limited to the at least one adaptive learning algorithm or another computational algorithm implemented at least as: executable software, digital hardware (e.g. FPGA, ASIC, custom chip design).

41. The method of claim 35, wherein the at least one adaptive learning algorithm includes, but not limited to at least one of the machine learning algorithms: a K-nearest neighbour algorithm, a regression analysis, ensemble tree based algorithms, maximum power point tracking, an artificial neural network, a deep convolutional neural network, a recurrent neural network, a reinforcement learning algorithm, random forest algorithm, a recommender system, genetic algorithm, Q-learning and a deep Q-learning algorithm, wherein the at least one of those or another computational algorithm is implemented in a closed-loop system.

42. The method of claim 35, wherein the method includes arranging the data processing arrangement to use, but not limited to the at least one adaptive learning algorithm or another computational algorithm to adjust iteratively the brain stimuli, so that electrical activity of the brain of the user is adjusted to an approximate target electrical activity of the brain as desired.

43. The method of claim 35, wherein the method includes using a control unit to receive input from at least one of the user or a third party device, wherein the control unit is communicably coupled with the data processing arrangement and includes a communication module for establishing a communication between the apparatus and the third party device.
